(19) United States
(12) Patent Application Publication
(10) Pub. No.: US 2005/0079221 A1
(43) Pub. Date: Apr. 14, 2005

(54) Ibuprofen narcotic composition and method for making same

(76) Inventors: Pieter Jurjen Groenewoud, Powder Springs, GA (US); John Weston Facemire, Douglasville, GA (US); Jayanthy Jayanth, Marietta, GA (US)

Correspondence Address: BRYAN W. BOCKHOP, ESQ.;ARNALL GOLDEN GREGORY LLP, 171 17TH STREET, SUITE 2100, ATLANTA, GA, 30363, US

(21) Appl. No.: 10/683,747

(22) Filed: Oct. 10, 2003

(51) Int. Cl. A61K 9/14; A61K 9/20; D04H 1/00

(52) U.S. Cl. 424/489; 264/109

(57) ABSTRACT

A pharmaceutical composition in a plurality of units includes ibuprofen, in a concentration of between 10% and 84%, by weight, of each of the plurality of units, and a narcotic analgesic, of which each of the plurality of units includes between 1 mg to 60 mg of the narcotic analgesic. Other ingredients include: a disintegrant, in a concentration of between 0.25% to 15% by weight of each of the plurality of units; a filler, in a concentration of between 2% to 90% by weight of each of the plurality of units; and a binder, in a concentration of between 0.25% to 20% by weight of each of the plurality of units.

### BACKGROUND OF THE INVENTION

1. Field of the Invention

The present invention relates to pharmaceutical compositions and, more specifically to a pharmaceutical composition of ibuprofen and a narcotic analgesic.

2. Description of the Prior Art

Ibuprofen is a non-steroidal anti-inflammatory agent commonly known to the pharmaceutical art. Many compositions of ibuprofen show poor tablet compression, poor stability and poor disintegration characteristics.

Ibuprofen tablets made from wet granulation methods may experience degraded dissolution over time. This may be due to sintering. One method used to reduce sintering is to increase the amount of excipients used in the compositions in order to isolate the ibuprofen particles. However, by increasing the concentration of excipients, problems arise in formulating high dose ibuprofen tablets because the tablets may become too large.

Solid dosage forms of non-steroidal anti-inflammatory agents in combination with narcotic analgesics are known and have been described as providing a synergistic therapeutic effect for the relief of pain. For example, diclofenac sodium and codeine phosphate tablets are made by mixing the diclofenac sodium and codeine with dicalcium phosphate, corn starch and colloidal silica. Another formulation is spray granulated with a solution of hydroxypropyl cellulose in deionized water. The dried granulation is mixed with carboxymethyl starch, colloidal silica and magnesium stearate. The resulting blend is compressed into tablets and film coated. One formulation for the treatment of pain uses a combination of ibuprofen and hydrocodone bitartrate.

In a wet granulation method for making various strength ibuprofen and codeine phosphate tablets, the ibuprofen is mixed with microcrystalline cellulose, calcium carboxymethylcellulose and fumed silica. This combination of material may be granulated using a solution of polyvinylpyrrolidone in isopropyl alcohol. The granules are sized, dried and blended with excipients such as calcium carboxymethylcellulose, fumed silica, stearic acid, and sodium metabisulphite.

Wet granulation of pharmaceuticals may be a relatively time consuming and complicated process. Therefore, wet granulated pharmaceuticals may be considered not to be relatively easy to manufacture.

Therefore, there is a need for a composition containing ibuprofen and a narcotic analgesic that is relatively easy to manufacture.

### SUMMARY OF THE INVENTION

The disadvantages of the prior art are overcome by the present invention which, in one aspect, is a pharmaceutical composition in a plurality of units (such as tablets or capsules) that includes ibuprofen, in a concentration of between 10% and 84%, by weight, of each of the plurality of units, and a narcotic analgesic, of which each of the plurality of units includes between 1 mg to 60 mg of the narcotic analgesic. Other ingredients include: a disintegrant, in a concentration of between 0.25% to 15% by weight of each of the plurality of units; a filler, in a concentration of between 2% to 90% by weight of each of the plurality of units; and a binder, in a concentration of between 0.25% to 20% by weight of each of the plurality of units. At least the ibuprofen and the narcotic analgesic are granulated in a dry compaction process.

In another aspect, the invention is a method of making a pharmaceutical composition. Ibuprofen, in a concentration of between 10% and 84%, a narcotic analgesic and at least one excipient are mixed in a dry powder phase. The ibuprofen, the narcotic analgesic and the at least one excipient are compacted to form a substantially dry compact material, also referred to as a compact. The dry compact material is milled, or otherwise sized, to form a plurality of dry granules. The dry granules are compressed to form a plurality of tablets.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a block diagram showing a process for making a pharmaceutical composition.

FIG. 2 is a table showing several compositions according to the invention.

### DETAILED DESCRIPTION OF THE INVENTION

A preferred embodiment of the invention is now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

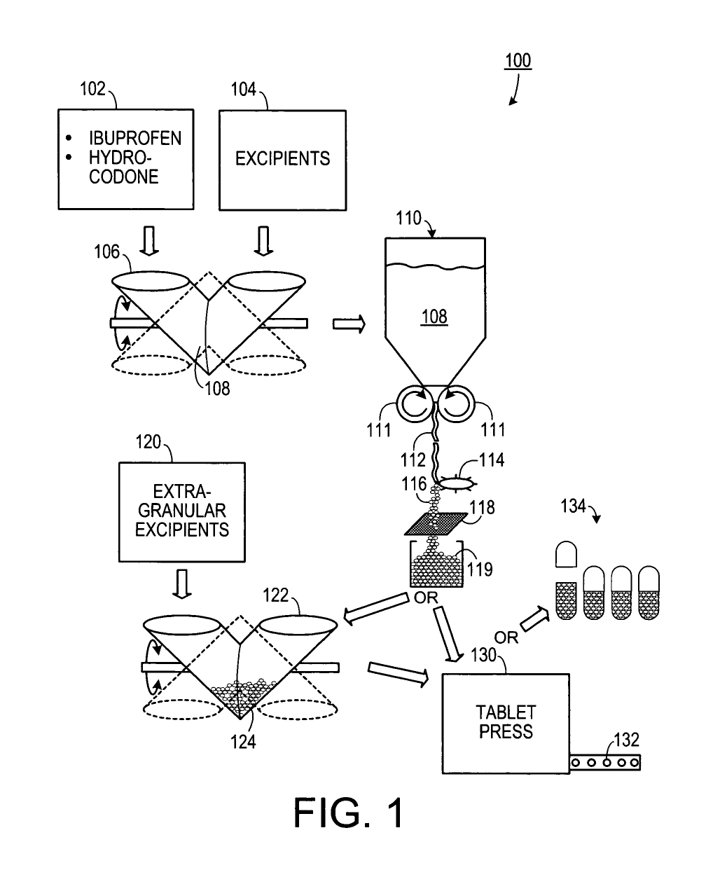

In one embodiment, as shown in FIG. 1, the invention is a process 100 for making a pharmaceutical composition of ibuprofen and a narcotic analgesic, such as hydrocodone bitartrate. In a powder phase, the active ingredients 102 are added to inactive excipients 104 and are mixed in a blender 106 to form a powdered combination 108. The powdered combination 108 is delivered to a dry compactor 110 where it is forced through a pair of compaction rollers 111 to form a plurality of compact materials 112. The compact materials 112 are milled into a plurality of granules 116 with a mill 114. The resulting granules 116 are screened 118. The screened granules 119 may be combined with extra-granular excipients 120 and mixed therewith in a blender 122 for form a combination 124 of granules and extra-granular excipients. The combination 124 is then delivered to a tablet press 130 which compresses the combination into a plurality of tablets 132. Alternately, capsule shells may be filled with the combination 124 to form a plurality of capsules 134.

If extra-granular excipients are not required, the screened granules 119 are delivered directly to the tablet press 130. Also, the ibuprofen may be granulated, as described above, with the hydrocodone bitartrate, or other narcotic analgesic being added extra-granularly.

The composition would include the following active ingredients: ibuprofen in a concentration of between 10% and 84%, by weight, of each of the plurality of tablets; and a narcotic analgesic, of which each of the plurality of tablets includes between 1 mg to 60 mg of the narcotic analgesic, such as hydrocodone bitartrate. The excipients may include: a disintegrant, such as croscarmellose sodium, in a concentration of between 0.25% to 15%, by weight, of each of the plurality of tablets; a filler, such as lactose and/or a filler/binder such as microcrystalline cellulose and lactose, in a concentration of between 2% to 90%, by weight, of each of the plurality of tablets; and a binder, such as pregelatinized starch, in a concentration of between 0.25% to 20%, by weight, of each of the plurality of tablets. A lubricant, such as magnesium stearate, in a concentration of about 0.59% by weight of each of the plurality of tablets may also be added. At least the ibuprofen and the narcotic analgesic are granulated in a dry compaction process.

Some illustrative excipients that may be used with the composition of the invention include the following: disintegrants (such as croscarmellose sodium, crospovidone, sodium starch glycolate, pregelatinized starch and starch), fillers and/or filler-binders (such as microcrystalline cellulose, cellulose, and lactose), binders (such as pregelatinized starch and povidone), and lubricants (such as magnesium stearate, sodium stearyl fumarate and stearic acid). As would be known to those of skill in the art, other excipients may also be employed.

The dry granulation process according to the invention has utility in that it offers the advantage of improved content uniformity of the composition. This feature is important with low-dosage pharmaceuticals, such as hydrocodone bitartrate. Another advantage of the invention is that it employs a relatively simple process requiring relatively few excipients.

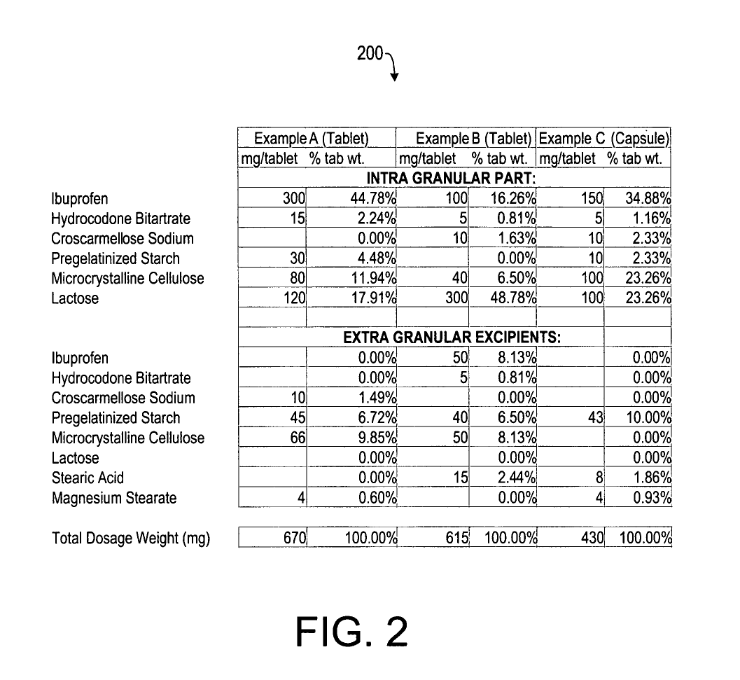

Several illustrative compositions according to the invention are shown in a table 200 in FIG. 2. These compositions include both tablet formulations and capsule formulations.

The above described embodiments are given as illustrative examples only. It will be readily appreciated that many deviations may be made from the specific embodiments disclosed in this specification without departing from the invention. Accordingly, the scope of the invention is to be determined by the claims below rather than being limited to the specifically described embodiments above.

1. A method of making a pharmaceutical composition, comprising the steps of:
(a) mixing, in a dry powder phase, ibuprofen, in a concentration of between 10% and 84%, a narcotic analgesic and at least one excipient;
(b) compacting the ibuprofen, the narcotic analgesic and the at least one excipient to form a substantially dry compact material; and
(c) milling the dry compact material to form a plurality of dry granules.

2. The method of claim 1, further comprising the step of compressing the dry granules to form a plurality of tablets.

3. The method of claim 1, further comprising the step of filling a plurality of capsule shells with the dry granules to form a plurality of capsules.

4. The method of claim 1, further comprising the steps of:
(a) adding at least one excipient to dry powder phase prior to the compacting step; and
(b) mixing the at least one excipient and the dry powder phase prior to the compacting step.

5. The method of claim 4, wherein the narcotic analgesic comprises hydrocodone bitartrate.

6. The method of claim 5, wherein the hydrocodone bitartrate is added in a concentration so that each of the plurality of tablets includes between 1 mg to 60 mg of hydrocodone bitartrate.

7. The method of claim 6, wherein the at least one excipient comprises croscarmellose sodium.

8. The method of claim 6, wherein the at least one excipient comprises microcrystalline cellulose.

9. The method of claim 6, wherein the at least one excipient comprises magnesium stearate.

10. A method of making a pharmaceutical composition, comprising the steps of:
(a) mixing, in a dry powder phase, ibuprofen and at least one excipient;
(b) compacting the ibuprofen and the at least one excipient to form a substantially dry compact material;
(c) milling the dry compact material to form a plurality of dry granules; and
(d) adding, extra-granularly, a narcotic analgesic to the dry granules, to form a pharmaceutical composition,
wherein the ibuprofen is in a concentration such that the pharmaceutical composition will have a concentration of ibuprofen of between 10% and 84%.

11. The method of claim 10, further comprising the step of compressing the pharmaceutical composition to form a plurality of tablets.

12. The method of claim 10, further comprising the step of filling a plurality of capsule shells with the pharmaceutical composition to form a plurality of capsules.

13. A pharmaceutical composition in a plurality of units, comprising:
(a) ibuprofen in a concentration of between 10% and 84%, by weight, of each of the plurality of units;
(b) a narcotic analgesic, of which each of the plurality of units includes between 1 mg to 60 mg of the narcotic analgesic;
(c) a disintegrant in a concentration of between 0.25% to 15%, by weight, of each of the plurality of units;
(d) a filler in a concentration of between 2% to 90%, by weight, of each of the plurality of units; and
(e) a binder in a concentration of between 0.25% to 20%, by weight, of each of the plurality of units,
wherein at least the ibuprofen and the narcotic analgesic are granulated in a dry compaction process.

14. The pharmaceutical composition of claim 13, wherein the narcotic analgesic comprises hydrocodone bitartrate.

15. The pharmaceutical composition of claim 13, wherein the disintegrant comprises croscarmellose sodium.

16. The pharmaceutical composition of claim 13, wherein the binder comprises microcrystalline cellulose.

17. The pharmaceutical composition of claim 13, wherein the filler comprises microcrystalline cellulose.

18. The pharmaceutical composition of claim 13, wherein the filler comprises lactose.

19. The pharmaceutical composition of claim 13, further comprising a lubricant in a concentration of about 0.59% by weight of each of the plurality of units.

20. The pharmaceutical composition of claim 19, wherein the lubricant comprises magnesium stearate.

21. The pharmaceutical composition of claim 13, wherein each of the units comprises a tablet.

22. The pharmaceutical composition of claim 13, wherein each of the units comprises a capsule.
